(19) United States
(12) Patent Application Publication
(10) Pub. No.: US 2008/0161760 A1
(43) Pub. Date: Jul. 3, 2008

(54) Medical Device Containing a Vacuum Chamber

(75) Inventor: Donald J Channer, Queensland (AU)

Correspondence Address: CANTOR COLBURN, LLP, 20 Church Street, 22nd Floor, Hartford, CT, 06103, US

(73) Assignee: MEDIGARD LIMITED, Main Beach (AU)

(21) Appl. No.: 11/660,020

(22) PCT Filed: Aug. 15, 2005
(86) PCT No.: PCT/AU05/01220

§ 371 Date: Feb. 8, 2007

(30) Foreign Application Priority Data
Aug. 20, 2004 (AU) 2004904738

(51) Int. Cl. A61M 5/19

(52) U.S. Cl. 604/229

(57) ABSTRACT

A needle containing medical device is provided with a mechanism to enable the needle to be sucked back into the main body of the device after use. The medical device contains a chamber that can be depressurised on demand to create the vacuum prior to use to enable the needle to be sucked back into the main body. Thus, the medical device can be stored and transported with the chamber under ordinary (atmospheric) pressure and the vacuum can be created on demand and just before use. Advantage—the medical device is not constantly under vacuum so the problem of slow loss of vacuum during storage is overcome.

### FIELD OF THE INVENTION

This invention is directed to a medical device, for instance a syringe, a catheter, a blood or body fluid collection container, generally of the type that has a puncture needle which is inserted into body tissue, and which has a vacuum chamber, and where the vacuum in the vacuum chamber can be generated on demand (typically just prior to use of the medical device). Thus, the medical device prior to use (for instance during storage or transportation) is not depressurised, and the vacuum is generated prior to use. The invention will be described generally with reference to a syringe, a catheter, or a blood collection container but it should be appreciated that no particular limitation is meant thereby.

### BACKGROUND ART

In the medical field, there are some instances where a partial vacuum is needed to operate a particular medical device. For instance, when collecting blood or other body fluid, it is usual to use a puncture needle which is inserted into the body tissue. A small container containing a rubber or other pierceable plunger/head member is provided and the container is under vacuum. The plunger is pushed up against the needle and the rear of the needle pierces the plunger. The vacuum inside the container then sucks body fluid (typically blood) into the container. This arrangement is widely used.

Ordinary syringes can also be used to remove fluids by retracting the plunger to create a partial vacuum which sucks blood/body fluid into the syringe body. This is also well known.

The dangers associated with needle-stick is well known and therefore there are many safety procedures in place to reduce the incidence of needle-stick injury. One known procedure is described in the applicant's earlier patent applications and is a mechanism that uses vacuum to shoot back (or more correctly, to suck back) a used needle into the main body of the syringe/device which contains the needle.

For instance, a syringe can be fitted or modified to suck back the needle once a syringe has been used. This is basically achieved by pushing the plunger body forwardly against the rear of the needle holder, and the plunger body and the needle holder has a special coupling/coupling arrangement such that the needle holder decouples from the front of the syringe barrel and attaches to a specially designed plunger head which decouples from the plunger body. The plunger body is under vacuum, and once the plunger head is decoupled from the plunger body, it is sucked back into the plunger body taking the needle holder (and the contaminated needle) with it. As mentioned above, this arrangement is subject to patent protection by the applicant.

Although the advantage of a shoot-back needle has been described with reference to a syringe in the previous paragraph, it should be appreciated that there are many other devices, particularly in the medical field, where there is an advantage in providing a shoot-back needle to minimise needle-stick injury. These devices may include a catheter and the like. There is an advantage in providing vacuum to facilitate the drawback of the needle as this does away with some of the disadvantages of providing a compressed spring etc. to provide the force necessary to retract the needle back into the main body of the device.

It is well known to provide a device with a chamber which is under vacuum. However, under certain situations, there may be an advantage if it were possible to provide a device which used vacuum to retract the needle, but where the vacuum was provided just before use, or shortly before use (or otherwise “on demand”). Therefore, there would be an advantage if it were possible to provide some sort of mechanism or arrangement that would enable a device to be depressurised prior to use as opposed to having a device which is always in the depressurised state.

It will be clearly understood that, if a prior art publication is referred to herein, this reference does not constitute an admission that the publication forms part of the common general knowledge in the art in Australia or in any other country.

### OBJECT OF THE INVENTION

It is an object of the invention to provide a device, and particularly a medical device of the type which can have a needle that might be prone to needle-stick injury, and where the needle can be retracted using vacuum, and where the vacuum can be provided in situ.

It is another object of the invention to provide a device, and particularly a medical device where a chamber can be depressurised “on demand”, for instance just prior to use.

In one form, the invention resides in a device and particularly a medical device which comprises a body adapted to be depressurised, the body containing an airtight chamber, one wall of the chamber comprising a member (which may be a sliding member) which is movable between a forward position to define a smaller volume in the chamber, and a rear position to define a larger volume in the chamber thereby producing a depressurised state in the chamber, and means to hold the member in the rear position.

In this manner, a vacuum can be formed in the airtight chamber just before use.

It is preferred that the means to hold the member in the rear position locks the member in the rear position.

In a simple form, the member may be a sliding member and may slide between the forward position and the rear position. However, it is also envisaged that the member can rotate, or otherwise move from the forward position to the rear position (e.g. by being threaded).

In a simple form, the device may comprise essentially a body containing an airtight chamber and the sliding member to form a vacuum just before use and the device can then be used to collect body fluid (e.g. blood) which can be sucked through a needle and into the chamber. Thus, the device can be a replacement for the conventional vacuum tubes.

However, in a more sophisticated form, the device can use the vacuum to retract the needle to prevent needle-stick injury. In this more sophisticated form, the chamber may have the sliding member defining one wall of the chamber, and may have a releasable locking member defining another wall of the chamber. The releasable locking member may be releasably locked to the device, and when released, will suck back into the chamber because of the vacuum in the chamber. The releasable locking member can contain the needle such that retraction of the releasable locking member will cause retraction of the needle. Alternatively, the releasable locking member may lock to a needle holder prior to being retracted into the chamber. These variations of the needle retraction mechanism are described in various of our earlier patent applications. However, in our earlier patent applications, the vacuum was not created just prior to use.

It is preferred that retraction means is provided to enable the sliding member to be moved from the forward position to the retracted position. In one form, the retraction means may comprise a line member which is attached to or relative to the sliding member and which can be accessed from outside the body such that pulling of the line member will cause the sliding member to move from the forward position to the vacuum creating retracted position.

The line member will preferably comprise a flexible and substantially inextensible line member and may comprise a plastic line member, a string, a metal line member, or a line member formed from any other suitable material. The term “line member” is meant to include a single line member, a braided line member, a wire, a rope, a chain, a strap, a strip and no unnecessary limitation should be placed on the term “line member”.

In another form, the retraction means may comprise a substantially rigid rod that may be attached to or relative to the sliding member and which can be retracted to retract the sliding member thereby creating the vacuum. The rigid rod may be made of any suitable material such as plastic, metal, composite materials and the like. The rod may be hollow or solid or substantially flat. The rod may have a cylindrical shape, or maybe rectangular, oval, or have any other regular or irregular shape. The rod may be provided with a “snap off” section, a frangible section, a breakable section and the like such that once the sliding member has been retracted, the retraction means can be removed.

It is preferred that the retraction means can be fully, or at least partially removed, or otherwise moved to not interfere with the normal operation of the device. It is preferred that the retraction means is substantially fully removed and disposed of. Therefore, it is preferred that the retraction means is removeably attached to, or relative to the sliding member such that when the sliding member has been retracted, the retraction means can be removed.

It is not considered that any unnecessary limitation should be placed on the present invention by any particular mechanism by which the retraction means can be removed. Thus, the retraction means can be pulled off the sliding member, twisted off, broken off, levered off or otherwise removed from the sliding member.

Suitably, the sliding member is provided with some form of engagement means to engage with the retraction means. The engagement means may comprise at least one recess in the sliding member into which a projection on the engagement means can fit. Alternatively, the engagement means may comprise a projection on the sliding member into which a recess on the engagement means can fit. Alternatively, the engagement means may comprise a projection on the sliding member and the retraction means which can engage with each other (e.g. hook together). As mentioned above, no particular limitation should be placed on the manner by which the retraction means is attached to the sliding member.

In a further alternative, the retraction means may comprise a projection on the sliding member that passes through an opening in the device such that the projection can be gripped to retract the sliding member. In this alternative, some form of seal may be required to prevent vacuum from being lost in the chamber.

The body may be of any suitable shape and configuration for its particular use. If the invention is to be used as a medical device to inject, or to remove fluid from body tissue, the body will typically have a length of between 5-40 centimetres. The body will typically be cylindrical in configuration and will typically have a diameter of between 5-40 millimetres. The body may be made of any suitable material such as plastic, metal, composite materials, glass and the like. The body may comprise a syringe barrel if the medical device is a syringe. The body will typically be substantially or essentially entirely hollow, although there may be situations where the body contains hollow sections and solid sections.

The body contains an airtight chamber. It is preferred that the airtight chamber substantially comprises the body to maximise the size of the airtight chamber. Thus, it is preferred that the body is substantially hollow. The airtight chamber will typically be cylindrical in configuration and will therefore typically have a side wall, a proximal end wall and a distal end wall (the distal end wall being the wall furthest away from the needle and the proximal end wall being closest to the needle). It is preferred that the sliding member comprises the distal end wall.

The sliding member may be of any shape and configuration providing that it can be retracted in the airtight chamber. For this reason, it is considered expedient for the sliding member to comprise a sliding sealing member such as a plunger. The sliding member may be made of any suitable material such as rubber, plastic and the like. The sliding member will typically be substantially cylindrical if the airtight chamber is substantially cylindrical; however if the chamber is oval or rectangular, the sliding member will then typically be oval or rectangular. The sliding member may be provided with extending sealing fins or ribs as is quite normal if the sliding member is a plunger. As mentioned above, the sliding member may be provided with some form of engagement means, such as a recess, to enable a retraction means to retract the sliding member. The sliding member can move along the airtight chamber to form a vacuum as the sliding member is retracted. Typically, the sliding member can move from adjacent a forward position where the sliding member is in the proximal part of the airtight chamber, towards a retracted position where the sliding member is in a distal part of the chamber. The sliding member will typically be slidable along a distance of between 1-20 centimetres and typically between 1-10 centimetres.

In one form of the invention, the airtight chamber contains a proximal end wall which contains a releasable member that can be releasably attached relative to the airtight chamber. The releasable member may be identical or similar to that described in our earlier patent applications. Thus, the releasable member may be attached directly to the needle, or the needle is directly attached to a needle holder and the releasable member can attach to, and retract the needle holder and therefore the needle.

In another form of the invention, the proximal end wall may comprise a pierceable member through which a “double ended” needle can pierce such that the vacuum in the chamber can suck body fluid through the needle and into the chamber. The pierceable member may comprise a rubber member, a plastic member and the like.

The device includes means to lock the sliding member in the rear position. In this “locked” position, the sliding member is prevented from moving back to the forward position to cause loss of vacuum. It is considered that any means to lock the sliding member in the rear position falls within the scope of the present invention. In one form, the sliding member may contain a part which can abut against an external part of the device once the sliding member has been fully retracted. The sliding member may be in a partially compressed state when sliding along the airtight chamber but when the portion of the sliding member exits from the airtight chamber, it can expand to lock against part of the device. The part may comprise one or more expandable fingers.

It is preferred that there is a cooperative-type arrangement between the retraction means and the locking of the sliding member in the rear position. The cooperative-type arrangement may be such that the retraction means causes a part of the sliding member to push outwardly to lock against the device, which at the same time, releases the retraction means from the sliding member. With this cooperative-type arrangement, the retraction means is released and prevents the sliding member from being pulled entirely out of the airtight chamber which is not preferred. Also, with this cooperative-type arrangement, the retraction means can cause expansion of the sliding member to cause the sliding member to lock against the device. Of course, no particular limitation should be placed on the invention merely by describing one mechanism by which the sliding member can be locked in the rear position.

### BRIEF DESCRIPTION OF THE DRAWINGS

Embodiments of the invention will be described with reference to the following drawings in which:

FIG. 1 illustrates a cross-section of a syringe containing a needle, a needle holder, a syringe barrel, and an internal plunger, the plunger containing an airtight chamber and a sliding member that can be retracted to create a vacuum in the chamber with the sliding member being in the forward “rest” position, and a retraction means in the form of a flexible line member.

FIG. 2 illustrates a close-up view of the sliding member in the forward “rest” position.

FIG. 3 illustrates the same syringe but where the sliding member has been pulled back to an almost fully retracted position to create a vacuum in the chamber.

FIG. 4 illustrates a close-up view of the sliding member in the almost fully retracted position.

FIG. 5 illustrates the same syringe where the sliding member has been pulled fully back to the “locked” position where the retraction means can now be removed without the sliding member moving back to the forward position.

FIG. 6 illustrates a close-up view of the sliding member in the fully retracted “locked” position.

FIG. 7 illustrates the syringe where the needle has been shot back into the plunger to prevent needlestick.

FIG. 8 illustrates a close-up view of the needle in the retracted position.

FIG. 9 illustrates an external view of a syringe containing the mechanism to enable vacuum to be formed in situ.

FIG. 10 illustrates a blood collection container containing the mechanism.

FIG. 11 illustrates a catheter/cannula containing the mechanism.

### BEST MODE

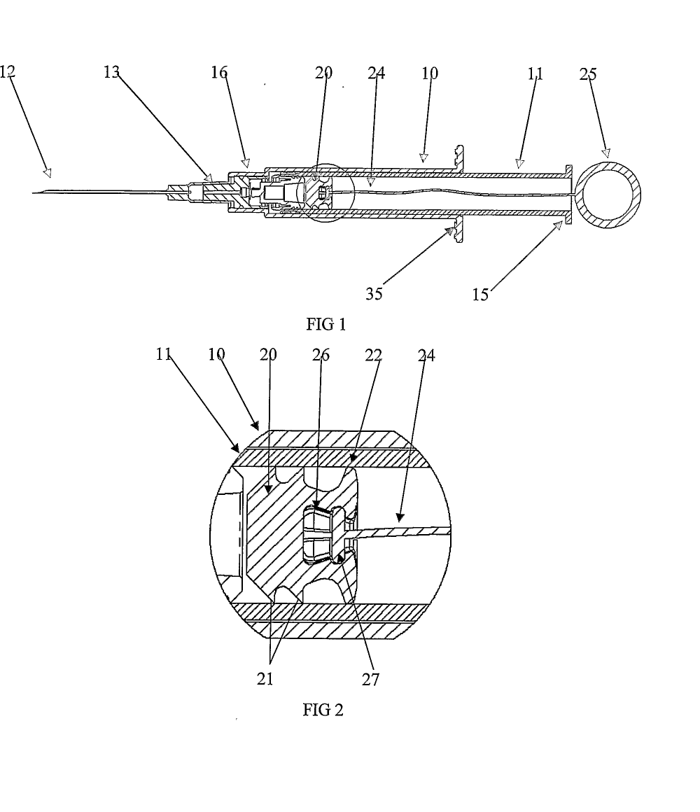

Referring initially to FIGS. 1-9, there is illustrated a medical syringe having a shoot-back needle, and where vacuum is used to assist in the retraction of the needle, and where the vacuum is created on demand using a particular mechanism that will be described below.

Referring initially to FIG. 1, there is illustrated a syringe which contains an external barrel 10, an internal plunger 11 that slides within the barrel, a needle 12 and a needle holder 13. The syringe will typically hold a fluid volume of between 1-50 millilitres.

Barrel 10 is cylindrical and hollow and contains a distal end which contains an outwardly extending annular flange 35 as is quite conventional. Flange 35 enables a person to push the plunger 11 forwardly by depressing the plunger with their thumb and attaching two fingers about flange 35. The rear of barrel 10 is open to enable plunger 11 to pass through the rear of barrel 10. This arrangement is quite conventional.

Barrel 10 has a proximal (forward) end which has a stepped down portion 16 defining a reduced diameter.

Needle 12 will typically comprise a conventional medical needle which is made of steel. Needle 12 has a forward pointed end for insertion into body tissue and a rear end. The rear end is fastened to a needle holder 13 which is positioned within the stepped down portion 16 in barrel 10. Needle holder 13 contains an internal passageway to accommodate needle 12. Needle holder 13 has a rear portion 17 best illustrated in FIG. 8. Rear portion 17 comprises a plurality of locking/unlocking fingers 18. Fingers 18, in the rest position, lock needle holder 13 to barrel 10 such that the needle holder cannot be retracted back into the barrel. However, needle holder IS can be unlocked from barrel 10 and attached to a second locking/unlocking member 19 which is positioned on the front of plunger 11.

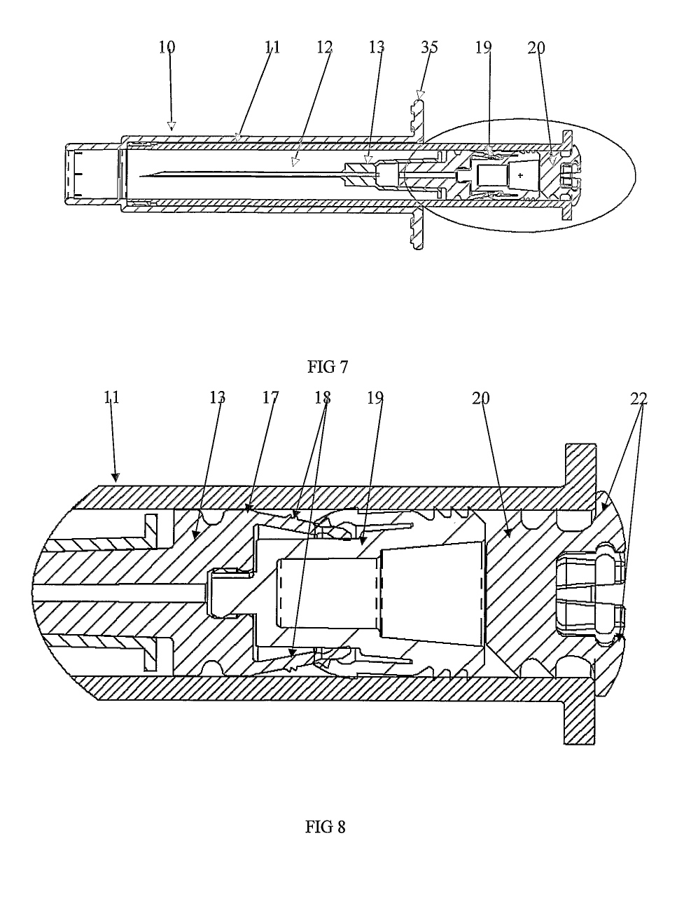

This arrangement is described in one or more of our previous patent applications but in summary, the arrangement enables needle 12 to be retracted into the inside of plunger 11 once plunger 11 has been fully pushed forward in barrel 10 such that the second locking/unlocking member 19, which is positioned on the front of plunger 11, abuts against rear portion 17, and the construction of member 19 and needle holder 13 is such that member 19 becomes unlocked from the front of plunger 11 and locked against needle holder 13 and, at the same time, needle holder 13 becomes unlocked from the inside of barrel 10. Plunger 11 is hollow and contains a vacuum, and as soon as member 19 becomes unlocked from the front of plunger 11, it will be sucked back into plunger 11 taking the needle holder and therefore the contaminated needle with it such that the needle is now protected inside the syringe as illustrated in FIG. 7.

In the present invention, there is illustrated a particular mechanism to enable a vacuum to be created within plunger 11 on demand, which will typically be prior to use.

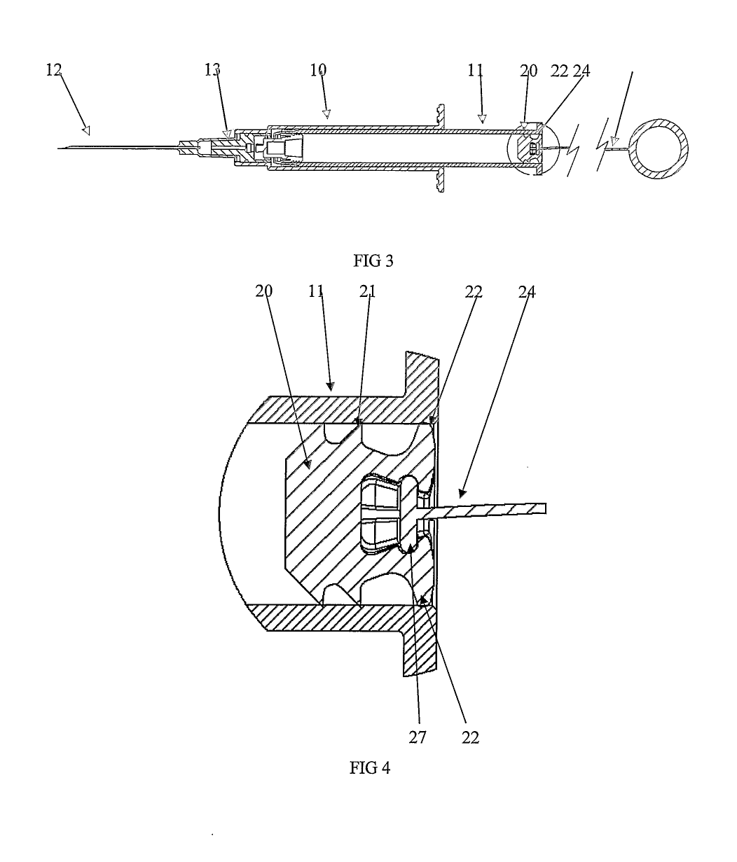

This is achieved by providing plunger 11 with a sliding member 20 which can slide from a proximal part of plunger 11 (see for instance the position of sliding member 20 in FIG. 1) to a distal position within plunger 11 (see for instance the position illustrated in FIG. 3 and FIG. 5). In doing so, the sliding member creates a vacuum within plunger 11. The front of plunger 11 is sealed by member 19 and therefore member 19 can be seen as one end wall of plunger 11 and sliding member 20 can be seen as the other end wall of plunger 11.

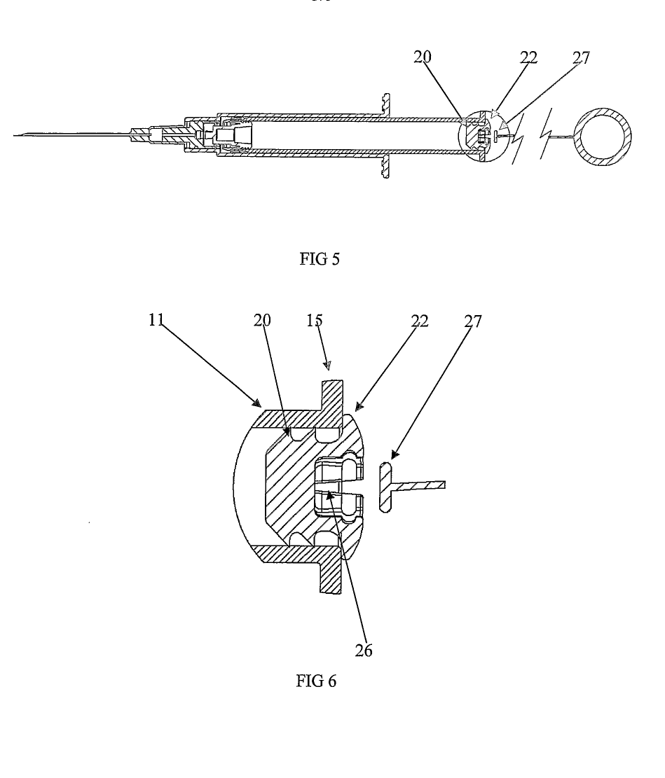

Sliding member 19 is better illustrated in FIG. 2 and comprises a plurality of sealing ribs 21 which sealingly engage against the inside wall of plunger 11. Sliding member 19 also contains a rear locking part 22 which comprises fingers that can move between a compressed state illustrated in FIG. 2 where the fingers are within the confines of plunger 11, and an expanded state (see FIG. 6 and FIG. 8) where the fingers abut against the outside of flange 15 on plunger 11. When the fingers are in this expanded position, sliding member 20 is locked in the retracted position and has created a vacuum within plunger 11.

Sliding member 19 can be retracted by a retraction means which in the particular embodiment comprises a line member 24. Line member 24 has a rear finger ring 25 to enable the line member to be easily gripped and pulled back. The forward part of line member 24 comprises a small plug or disk 27 (best illustrated in FIG. 6). Disk 27 sits within a recess 26 within sliding member 20.

Prior to use, the syringe will be in the position illustrated in FIG. 1 which is where the sliding member 20 is in a forward part of plunger 11. When vacuum is required, finger ring 25 is gripped and pulled back to pull sliding member 20 to the distal part of plunger 11 creating a vacuum in the process. When the sliding member has been almost fully retracted (see FIG. 3 and FIG. 4), the locking part 22 of sliding member 20 is just about to exit the rear of plunger 11. Disk 27 is held behind the locking part (fingers) of sliding member 20 and cannot be released from the sliding member because the locking part is compressed by being within the confines of plunger 11. Slight further retraction brings sliding member 20 to the position illustrated in FIG. 6 where the locking part (fingers) has been pulled out of the rear of plunger 11. As soon as this occurs, further pulling of the line member 24 causes disk 27 to be pulled out of engagement with sliding member 20 and this process also ensures that the fingers (locking part 22) abut against the outside of the flange 15 on the rear of plunger 11 to securely lock the sliding member in the retracted position. Thus, plunger 11 now contains a vacuum.

Forward movement of plunger 11 through barrel 10 will ultimately cause member 19 to engage with needle holder 13 and the vacuum ensures that member 19 and the attached needle holder is sucked back into the confines of plunger 11 to the position illustrated in FIG. 7 and FIG. 8.

Thus, the present invention allows the vacuum in plunger 11 to be created when required which will typically be just prior to use. Creation of the vacuum is extremely simple and merely requires the line member 24 to be pulled to cause sliding member 20 to retract along plunger 11 and ultimately to be locked against the back of plunger 11.

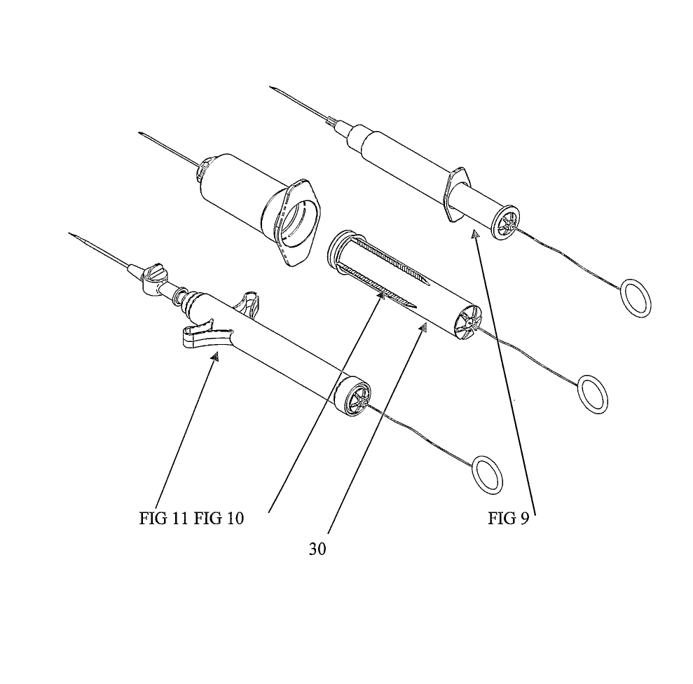

FIG. 9 is an external view of a single-use syringe. FIG. 10 is an external view of the blood collection device which contains a vacuum chamber 30 where vacuum can be created in vacuum chamber 30 just prior to use and using a mechanism as described above. FIG. 11 illustrates a medical device which can retract the puncture needle in a catheter and which has been described in a previous application but now where the vacuum can be created on demand.

Throughout the specification and the claims (if present), unless the context requires otherwise, the term “comprise”, or variations such as “comprises” or “comprising”, will be understood to apply the inclusion of the stated integer or group of integers but not the exclusion of any other integer or group of integers.

Throughout the specification and claims (if present), unless the context requires otherwise, the term “substantially” or “about” will be understood to not be limited to the value for the range qualified by the terms.

It should be appreciated that various other changes and modifications can be made to any embodiment described without departing from the spirit and scope of the invention.

1. A device comprising a body adapted to be depressurised, the body containing an airtight chamber having a front opening, a releasable seal to seal the front opening, one wall of the chamber comprising a member which is movable between a forward position closer to the front opening to define a smaller volume in the chamber, and a rear position further from the front opening to define a larger volume in the chamber thereby producing a depressurised state in the chamber, and means to hold the member in the rear position.

2. The device as claimed in claim 1, including a retraction member which is adapted for retraction into the depressurised chamber, the retraction member being releasably coupled relative to the device, and means to release the retraction member such that the retraction member can be sucked back into the depressurised chamber.

3. The device as claimed in claim 2, comprising a medical device, the body comprising a plunger, and the retraction member comprising a needle.

4. The device as claimed in claim 3, wherein the wall of the chamber slides between the forward position and the rear position.

5. The device as claimed in claim 4, including retraction means to retract the member from the forward position to the rear position.

6. The device as claimed in claim 5, wherein the retraction means can be removed from the device when the member has been moved to the rear position.

7. The device as claimed in claim 5, wherein the retraction means comprises a line member which is attached to the movable member, the retraction means able to be pulled back to pull the movable member from the forward position to the rear position.

8. The device as claimed in claim 6, wherein the plunger comprises a front sealed area and the sliding member which is adapted for movement between a forward position where the sliding member is adjacent the front sealed area and the rear position where the sliding member is retracted along the plunger to create a vacuum between the front sealed area and the sliding member.

9. The device as claimed in claim 8, wherein the front sealed area is releasably attached to the front of the plunger, and contains attachment means adapted to attach to the needle or needle holder when the plunger is pushed towards the needle or needle holder.

10. The device as claimed in claim 7, wherein the movable member is provided with a recess, the line member being provided with a plug, the plug being insertable into and held in the recess when the movable member moves from the forward position towards the rear position, the recess having an opening which is larger than the plug such that the plug can be released from engagement with the recess, the opening being compressed into a smaller size when the movable member is in the plunger, the smaller size holding the plug in the recess, wherein when the movable member is in the rear position, part of the member extends from the rear of the plunger thereby allowing the opening to return to the larger size, thereby allowing the plug to be pulled out of the opening.

11. A medical device which contains an outer body, a plunger that can slide within the outer body between a retracted position and a forward position, the outer body containing an open rear end into which the plunger can be placed, and a forward end, a needle assembly in the forward end, the needle assembly being releasably attached relative to the outer body, the plunger having a forward end containing a releasable sealing member and containing means to engage with the needle assembly when the plunger is pushed towards the forward end of the outer body, the plunger further having a movable member which is in sealing engagement inside the plunger and which is movable between a forward position where the movable member is adjacent the releasable sealing member and a rear position where the movable member has been retracted along the plunger towards a rear end of the plunger thereby creating a vacuum in the plunger between the sealing member and the movable member, retraction means attachable to the movable member to enable the movable member to be retracted, whereby vacuum can be created in the plunger prior to use by retracting the movable member, and release means to release the sealing member from the front of the plunger to cause the sealing member to be retracted into the plunger under vacuum, and means to attach the sealing member to the needle holder such that retraction of the sealing member into the plunger also causes retraction of the needle holder into the plunger.

12. A medical device containing a plunger and a needle, and where the plunger contains a chamber that is at atmospheric pressure, but which can be depressurised on demand.
